(19) United States
(12) Patent Application Publication
(10) Pub. No.: US 2004/0172200 A1
(43) Pub. Date: Sep. 2, 2004

(54) Constellation mapping and uses thereof

(76) Inventors: Paul Edward Kearney, Montreal (CA); Navdeep Jaitly, Montreal (CA); Kevin Eng, Montreal (CA)

Correspondence Address: BOZICEVIC, FIELD & FRANCIS LLP, 200 MIDDLEFIELD RD, SUITE 200, MENLO PARK, CA, 94025, US

(21) Appl. No.: 10/719,916

(22) Filed: Nov. 21, 2003

Related U.S. Application Data
(60) Provisional application No. 60/428,731, filed on Nov. 22, 2002.

(51) Int. Cl. G06G 7/48; G06G 7/58; G06F 19/00; G01N 33/48; G01N 33/50

(52) U.S. Cl. 702/19; 702/20

(57) ABSTRACT

The present invention features computer methods and systems for comparing biomolecules across biological samples. In these methods, mass spectrometry measurements are obtained on biomolecules in two or more samples. These measurements are then processed and analyzed by the methods described herein to render them more comparable. We refer to this technology as “Constellation Mapping” (CM). The resulting data, constellation maps, can be used to compare the abundance of biomolecules across samples, and, when done in real time, can be used to select differentially abundant biomolecules for subsequent LC/MS-MS.

### RELATED APPLICATIONS

This application claims priority to the U.S. Provisional Application Serial No. 60/428,731, filed Nov. 22, 2002, the disclosure of which application is hereby incorporated in its entirety by this reference.

### FIELD OF THE INVENTION

The invention relates to the fields of mass spectrometry, bioinformatics, and computational molecular biology. In particular, this invention relates to the comparison of biomolecule abundance for two or more samples.

### BACKGROUND OF THE INVENTION

Genomic and proteomic research efforts in recent years have vastly improved our understanding of the molecular basis of life at a global cellular and tissue scale. In particular, it is increasingly clear that the temporal and spatial expression of an organism's biomolecules is responsible for life's processes—processes occurring in both health and in sickness. Science has progressed from understanding how genetic defects cause hereditary disorders, to an understanding of the importance of the interaction of multiple genetic defects together with environmental factors in the etiology of complex medical disorders, such as cancer. Scientific evidence demonstrates the key causative roles of altered expression of, and multiple defects in, several pivotal genes and their protein products in human cancer. Other complex diseases have similar molecular underpinnings. Accordingly, the more complete and reliable a correlation that can be established between expression of an organism's biomolecules and healthy or diseased states, the better diseases can be diagnosed and treated. Methods that permit efficient and rapid comparison of biomolecule expression from different biological samples that may each contain tens of thousands of biomolecules (e.g., protein, lipids, nucleic acids, carbohydrates, metabolites, and combinations thereof) are necessary to provide the best possible chance to determine such correlations. For example, proteomic data reflects the true expression levels of functional molecules and their post-translational modifications, which cannot be accurately predicted from other data types such as gene expression profiling.

A central goal of proteomics, which involves the systematic identification and characterization of proteins in a sample, is to be able to compare the protein composition between two or more samples. Critical to achieving this goal is the ability to identify all the proteins that are present in only one sample or type of sample and any proteins that are present in several samples or types of samples but differ in abundance.

The methods by which two biomolecules are judged to be the same or comparable depend on the methods employed to identify a particular biomolecule within a field of biomolecules and the completeness of the data gathered from a sample. Presented with a the full range of proteins from a tissue sample, however, identifying and matching proteins for comparison can be extremely problematic. Usually, a comparison of all the proteins in a sample is accomplished by two-dimensional (2D) gel electrophoresis, which resolves a complex protein mixture into hundreds or thousands of spots, which have characteristic migratory positions for particular proteins. Gel patterns can be directly comparable with correction of migration variables if the gel and sample were properly prepared and run, but gel reproducibility is quite variable from lab to lab or even with different lots of ampholines. Each spot, in theory, represents one protein, and the intensity of each spot is taken as a measure for the amount of the protein present. The protein that is present in this spot can then be more fully identified by mass spectrometry or other methods; however, the further identification of a single protein spot, let alone the whole field of spots, can involve considerable time, effort, and expense. The 2D electrophoresis approach also has several other drawbacks, the most important of which is the difficulty of identifying membrane proteins. In general, 2-D electrophoresis has problems with the exclusion of highly hydrophobic molecules, and with the detection of highly charged (very acidic or very basic) molecules, as well as of very small or very large molecules. In addition, the detection of low or even moderate abundance proteins is difficult and may require that several gels be run to collect enough material for sequence analysis. 2D gel spots can also be quite large, which dilutes the protein over a large part of the gel, rendering detection and accurate quantification of proteins more difficult. Additionally, co-migration of proteins, particularly of closely related or variant proteins, can interfere with both proper identification and quantification of the specific proteins.

One-dimensional (ID) gel electrophoresis, on the other hand, is a generally applicable tool to separate proteins that at least allows the study of both soluble and membrane proteins. However, when complex mixtures of proteins are analyzed, only 50 to 100 protein bands are typically detectably produced in the separation, and a single band in a 1D gel may, therefore, contain more than a single protein. For this reason, the intensity of one band does not typically reflect the abundance of a single protein in the sample, and identification likewise becomes more problematic. Mass spectrometry, for example, of a single band will lead to the identification of not just one but several (e.g. 10 to 20) proteins that are present in the band at different concentrations.

Mass spectrometry itself is a method of choice for analyzing complex mixtures of molecules, such as the contents of cells, or cellular components. When combined with appropriate methods of chromatography to allow separation and purification of biomolecules, mass spectrometry provides a start point for producing and analyzing data for the identification and quantification of biomolecules, and for patterns that liken or distinguish different samples.

At its most basic, mass spectrometry produces data about the mass of biomolecules, and their intensity (ion counts) for a particular scan. Fragmentation patterns for specific molecules can also be produced, but these characteristic spectra, which can be used to further identify the molecule, are unlinked to the quantitative data (ion counts) produced in the initial scan. Secondary efforts are required to derive structural information from this basic data, or, in the case of polymers such as DNA or proteins, to obtain sequence information from the fragmentation patterns, to determine the source protein from the sequence information, and to couple sequence/identity information to quantification data.

One quantitative mass spectrometric technique relies on coupling different isotopic tags to the peptides of each sample to be analyzed. An example of this methodology is referred to as isotope-coded affinity tag (ICAT) (see Han et al. (2001) Nat. Biotechnol. 19: 946-51 (PMID: 11581660)). This method consists of derivatizing proteins, such as with alkylating agents containing a reactive group specific to cysteine residues, a linker chain, and a biotinylated moiety. The alkylating agent includes a light and a heavy version corresponding to 8 hydrogen atoms (light) or 8 deuterium atoms (heavy) in the linker chain. When comparing two samples, all the peptides from one sample are tagged with the light tag, and all the peptides from the other sample are tagged with the heavy tag. Both samples are then mixed, digested with trypsin, and analyzed simultaneously. In the mass spectrum, ions pairs that correspond to the same peptide but differ by the exact mass difference (8 Da) between the heavy and light tag are then identified. These ions then correspond to the same peptide but are derived from the two different samples. This method allows for the direct comparison of the abundance of corresponding peptides from the two samples. Despite permitting direct comparison of samples, this technique generally has the limitation that all peptides containing cysteine residues must be chemically modified before they are analyzed. Such modifications come at an additional expense in both money and time. They can also have a cost in accuracy if the reaction does not go to completion, or the delays due to processing time lead to protein degradation. Furthermore, the chemical modification requires the presence of a specific amino acid, cysteine, in the peptide, which means that the majority of peptides are not suitable for the analysis. This requirement greatly reduces the applicability of this approach to a wide range of proteins. The ICAT approach can also generate interfering intensities from biotinylated fragment ions in MS/MS experiments, hampering the ability to determine peptide sequence information.

Another labeling method uses light and heavy isotopes of water. Tryptic peptides from different protein pools are labeled at the C-terminus with ¹⁶O and ¹⁸O water. This method has been used to distinguish between b- and y-type fragment ions in MS/MS experiments (see Schevshenko et al. (1997) Rapid Commun. Mass Spectrom. 11: 1015-1024). The method has also been used for monitoring the differential expression of proteins in two serotypes of adenovirus (see Yao et al. (2001) Anal. Chem. 73: 2836-2842). As above, protein pools are digested separately, labeled, and combined for analysis by mass spectrometry. Expression profiles are then obtained based on the ratio of heavy to light ions. This method also requires that the peptides or proteins be labeled before analysis, and thus, like ICAT may suffer from incomplete reactions, substrate insusceptibility, extra cost, and extra preparation time made all the more costly by the possible detriment to limited and potentially unstable samples. These issues are exacerbated by the additional challenges of preparing such samples from living organisms.

Methods making use of mass spectrometry data may rely on theoretical or predicted retention times for biomolecules to identify and compare the constituent biomolecules of two or more samples. Such methods may circumvent the need for derivatizing or labeling samples prior to mass spectrometry, but can suffer from error that can result in false positives and false negatives, limiting the accuracy of the comparison, hampering its validation, and slowing the process. The variability between samples induced by even minimal changes in instrument properties, such as the flow rate of a chromatography column are not readily predictable and can also exacerbate error.

Existing methods for comparison of the biomolecules present in mass spectrometric data are therefore in need of improvement in their ability to perform rapid, accurate, automated, and economical as well as qualitative, quantitative, and specific determinations of the components of a biological sample. For example, there exists a need for improved methods using mass spectrometric data to compare the abundance of peptides in samples containing peptides that have not been chemically modified prior to spectrometry, and that minimize sample variability. Furthermore, there is a continuing and significant need to be able to readily compare the relative abundances of proteins between biological samples, and to identify and characterize proteins as targets for drug discovery. The present invention fulfills these needs and further provides other related advantages.

### SUMMARY OF THE INVENTION

The present invention features computer methods and systems for comparing biomolecules across biological samples. In these methods, mass spectrometry measurements are obtained on biomolecules in two or more samples. These measurements are then processed and analyzed by the methods described herein to render them more comparable. We refer to this technology as “Constellation Mapping” (CM). The resulting data, constellation maps, can be used to compare the abundance of biomolecules across samples, and, when done in real time, can be used to select differentially abundant biomolecules from LC-MS scans for subsequent LC/MS-MS acquisition. LC/MS-MS spectra results can be used to identify biomolecules, such as peptides and proteins. This CM technology for permits rapid and accurate identification of individual biomolecules whose presence, absence, or altered expression is associated with a disease or a condition of interest. Such biomolecules (for example, proteins) are potentially useful as therapeutic agents, as targets for therapeutic intervention, or as markers for diagnosis, prognosis, and evaluating response to treatment. CM technology also permits rapid identification of sets of biomolecules whose pattern of expression is associated with a disease or condition of interest; such sets of biomolecules provide a collection of biological markers for potential use in diagnosis, prognosis, and evaluating response to treatment.

In one aspect, the invention features a method for determining an abundance of a biomolecule in a biological sample. In general, the method includes the steps of providing a biological sample containing a plurality of biomolecules; generating a plurality of ions of the biomolecules; performing mass spectrometry measurements on the plurality of ions, thereby obtaining ion counts for the biomolecules; assigning an ion to a biomolecule; and integrating the ion counts of the biomolecule, thereby determining the abundance of the biomolecule in the biological sample. Abundance calculations may be similar to those used for MIPS (“Mass Intensity Profiling System and Uses Thereof”, U.S. Utility patent application Ser. No. 10/293,076).

In particular, the invention features methods and systems for determination and comparison of the abundance of peptides in two or more samples, but the following methods may be applied to other biomolecules as well. These methods are based on the analysis of data from mass spectrometry, which may come from one or more LC/MS scans.

The invention also allows for the rapid matching of a biomolecule from an LC-MS scan with its corresponding LC-MS/MS fragmentation spectra, if acquired. For peptides, for example, this permits the coupling of LC-MS/MS based sequence data with peptide abundance data.

In another embodiment, CM can be used to query the abundance of one or more peptides or proteins in one or more samples, with or without prior calculation of said abundances, and with or without prior identification of the one or more peptides or proteins.

In various embodiments, the calculation of peptide abundance may be absolute or relative. In general, abundance is determined by a sum of ion counts based on a consistent choice within a sample, for example, a subset of charge states, isotopes, modified states, or a combination thereof.

Sample data need not be newly generated. One or more of the sets of data used for comparison may be from within the same set of sample data, and/or from one or more other sets of data including, but not limited to, reference, manipulated, representative, combined, and/or theoretical samples. The data need not be processed from scratch, but may pick up processing at an intermediate level, such as from an isotope map or peptide map. Comparisons may be part of iterative or cumulative processes.

In various embodiments, a peptide or protein in a sample may be used as the whole or part of the generation of a list of one or more peptides or proteins, which may in turn be combined with other lists or used directly or indirectly for querying, matching, or governing data gathering, such as selection for spectra determination by LC/MS-MS in further analysis of the same or another sample.

The invention further features a computer implemented method for comparing the abundance of biomolecules between two or more biological samples. The computer implemented method generally includes the steps of inputting mass spectrometry data, centroiding and reducing the noise, producing isotope maps, detecting and centering peptides, producing peptide maps, and aligning peptide maps, thereby allowing the determination of differential abundance of biomolecules in the biological samples.

In general, the invention features a computer-readable memory that comprises one or more programs for comparing the abundance of biomolecules between two or more biological samples, comprising the steps of inputting mass spectrometry data, centroiding and reducing the noise, producing isotope maps, detecting and centering peptides, producing peptide maps, and aligning peptide maps, thereby allowing the determination of differential abundance of biomolecules in the biological samples.

In yet another aspect, the invention includes an embodiment, wherein the system includes a processor and a memory coupled to the processor, wherein the memory encodes one or more of the following: a noise reduction module, a peptide detection module, and/or a peptide map alignment module.

In another aspect, the invention features a method for displaying information on abundance of a biomolecule in a biological sample to a user comprising the steps of inputting mass spectrometry data comprising ion counts for a plurality of biomolecules; assigning an ion to a biomolecule; integrating the ion counts of the biomolecule, thereby determining the abundance of the biomolecule in the biological sample; and displaying the abundance of the biomolecule. In one embodiment, the method can further include storing the abundance of the biomolecule in a memory.

In various embodiments of any of the aforementioned aspects, the biomolecule may be underivatized and/or unlabeled. The biomolecule may also be cleaved biomolecule. In preferred embodiments, the biomolecule is cleaved with an enzyme. In general, however, the methods do not require modification other than cleavage, such as isotope-labeling or akylation, of the biomolecules, i.e., cleaved biomolecules may be underivatized and/or unlabeled. The invention, if desired, features the inclusion of one or more internal standards in the biological sample.

In still another embodiment, a computer procedure assigns the ion to the biomolecule by calculating an uncharged mass for the ion. Alternatively, ions may be assigned to biomolecules through mass fingerprinting, e.g., peptide mass fingerprinting. In yet another embodiment, a computer procedure integrates ion counts of the ions corresponding to the biomolecule. Preferably, the integration is over one or more charge states, isotopes, scans, fragments of the biomolecule, fractions of a separation, or a combination thereof. In other embodiments, the invention further features separating the plurality of biomolecules prior to MS analysis. Typically, such separation is carried out using standard methods known in the art. These methods include, without limitation, chromatography, electrophoresis, immunoisolation (e.g., using magnetic beads), or centrifugation. The retention time of an ion may be corrected using one or more internal standards.

In various other embodiments of any of the aforementioned aspects, the biomolecule is typically a protein or modified protein. Preferably, the protein is obtained from an isolated organelle. Exemplary isolated organelles include, without limitation, mitochondria, chloroplasts, ER, Golgi, endosomes, lysosomes, phagosomes, peroxisomes, secretory vesicles, transport vesicles, nuclei, and plasma membrane. Proteins obtained from other cellular components are also useful in the invention. These proteins include cytosolic or cytoskeletal proteins.

In preferred embodiments, mass spectrometry measurements are obtained to gather structural or sequence information of an ion of the biomolecule, e.g., through MS/MS analysis. Biomolecules or ions thereof may be selected for structural or sequence analysis (e.g., MS/MS analysis) by a query. In one embodiment, an inclusion or exclusion list is used to determine which ions will be subjected to structural or sequence analysis. The methods and systems of the invention further feature the use of a computer procedure to identify a protein comprising the sequence of the ion from a database. Exemplary procedures include Mascot®, Protein Lynx Global Server, SEQUEST®/TurboSEQUEST, PEPSEQ, SpectrumMill, or Sonar MS/MS. Exemplary databases that are searched using such procedures include the Genbank®, EMBL, NCBI, MSDB, SWISS-PROT®, TrEMBL, dbEST, or Human Genome Sequence database. Moreover, the methods and systems include a computer procedure that assigns the ion to the protein identified from a database.

In various other embodiments of any of the aforementioned aspects, the invention features calculating an abundance of the biomolecule relative to a control biological sample and calculating abundances of a plurality of the biomolecules relative to a control biological sample. Typically, abundance measurements of a set of biomolecules are used to diagnose a disease or condition. Additionally, abundance is used to determine a biomolecule to target with a drug. Such targets are identified by evaluating an increase or decrease in abundance or the presence or absence of a biomolecule in the biological sample relative to a control sample. Abundance of a biomolecule may also be used to determine an amount of an isoform of a biomolecule, or of a naturally occurring modification of a biomolecule.

By “assigning an ion to a biomolecule” is meant specifying a biomolecule from which an ion observed in a mass spectrum was generated. The ion may be assigned to a biomolecule or a fragment thereof. Such assignments may be based, for example, on the molecular mass, or other physicochemical characteristic. The assignment can also be made on the basis of determining the molecular mass of the ion and matching that mass with a known biomolecule or on the basis of data, e.g., from MS/MS, that identifies structural or sequence information about the ion, which may be used to search a database.

By “biomolecule” is meant any organic molecule that is present in a biological sample, including peptides, polypeptides, proteins, post-translationally modified peptides or proteins (e.g., glycosylated, phosphorylated, or acylated peptides), oligosaccharides, polysaccharides, lipids, nucleic acids, and metabolites. Biomolecules may be in their natural state, isolated, purified, labeled, derivatized, cleaved, fragmented, combinations thereof, and the like. Preferably biomolecules are unlabeled or underivatized. More preferably they are unlabeled and underivatized. Preferably the biomolecules are proteins and peptides, and more preferably they are cleaved with a protease, preferably trypsin.

By “biological sample” (or “sample”) is meant any solid or fluid sample obtained from, excreted by, or secreted by any living organism, including single-celled micro-organisms (such as bacteria and yeasts) and multicellular organisms (such as plants and animals, for instance a vertebrate or a mammal, and in particular a healthy or apparently healthy human subject or a human patient affected by a condition or disease to be diagnosed or investigated). A biological sample may be a biological fluid obtained from any location (such as blood, plasma, serum, urine, bile, cerebrospinal fluid, aqueous or vitreous humor, or any bodily secretion), an exudate (such as fluid obtained from an abscess or any other site of infection or inflammation), or fluid obtained from a joint (such as a normal joint or a joint affected by disease such as rheumatoid arthritis). Alternatively, a biological sample can be obtained from any organ or tissue (including a biopsy or autopsy specimen) or may comprise cells (whether primary cells or cultured cells) or medium conditioned by any cell, tissue or organ. If desired, the biological sample is subjected to preliminary processing, including preliminary separation techniques. For example, cells or tissues can be extracted and subjected to subcellular fractionation for separate analysis of biomolecules in distinct subcellular fractions, e.g., proteins or drugs found in different parts of the cell. A sample may be analyzed as subsets of the sample, e.g., bands from a gel.

“CM” refers to Constellation Mapping.

By “fraction” is meant a portion of a separation. A fraction may correspond to a volume of liquid obtained during a defined time interval, for example, as in LC (liquid chromatography). A fraction may also correspond to a spatial location in a separation such as a band in a separation of a biomolecule facilitated by gel electrophoresis.

“Injections” refer to injections on a mass spectrometer, from which measurements can be made.

By “integrating the ion counts of a biomolecule” is meant summing ion counts for data within a defined range of m/z values. The phrase also refers to summing integrated ion counts of two or more ions. For example, ions that are found in different charge states, isotopes, fractions of a separation, scans, or fragments of a biomolecule may be integrated.

“Intensity normalization” refers to an adjustment of intensity values in one or more sets of data generally by linear regression, which can permit more relevant comparison between data sets, such as an the calculation of peptide abundance via MIPS (“Mass Intensity Profiling System and Uses Thereof”, U.S. Utility patent application Ser. No. 10/293,076).

“LC” refers to liquid chromatography.

“LC-MS” or “LC-MS” refers to liquid chromatography coupled with mass spectrometry, as is known in the art.

“LC-MS-MS” or “LC-MS/MS” refers to liquid chromatography couple with tandem mass spectrometry, as is known in the art.

“MS-MS” or “MS/MS” refers to tandem mass spectrometry as is known in the art.

By “precursor” is meant a biomolecule, e.g., a potential peptide or protein or one of unknown sequence or identity. Generally it refers to potential peptides in mass spectrometry survey scan data prior to secondary identification efforts, such as sequencing by MS/MS.

“Precursors” are frequently identified by comparing their masses or their retention times. Such retention times may be experimental or theoretical. Theoretical retention times are frequently corrected, where one or more internal standards are used to make retention times comparable between samples. Predicted retention times may be used to seek precursors within a scan. “Precursor” is frequently used interchangeably with “peptide,” and it may be used to distinguish individual constituent peptides from full-length proteins.

By the term ‘protein” is meant any polymer of two or more individual amino acids linked via a peptide bond that forms when the carboxyl carbon atom of the carboxylic acid group bonded to the alpha-carbon of one amino acid (or amino acid residue) becomes covalently bound to the amino nitrogen atom of amino group bonded to the alpha-carbon of an adjacent amino acid. The term “protein” is understood to include the terms “polypeptide” and “peptide” (which, at times, may be used interchangeably herein) within its meaning, as well as post-translational modifications and fragments thereof. It may be singular or used collectively, and may also refer to multiple isoforms, variants, modifications, related family members, and the like. In addition, proteins comprising multiple polypeptide subunits (e.g., insulin receptor, cytochrome b/c1 complex, and ribosomes) or other components (for example, an RNA molecule) will also be understood to be included within the meaning of “protein” as used herein. Similarly, fragments of proteins and polypeptides are also within the scope of the invention and may be referred to herein as “proteins,” “polypeptides,” or “peptides,” “tryptic peptides”, or “cleavage fragments.” “Constituent peptides” are peptides whose sequence is a linear subset of the sequence of a larger peptide or full-length protein. As a group, the “constituent peptides” for a particular protein would be a set or subset of those that make up the protein. Usually, this is a subset limited to particular cleavage fragments, such as the set of tryptic peptides that make up a protein. A “full-length protein” refers to a protein encoded by and translated from a messenger RNA (mRNA), and post-translational modifications thereof. Full-length proteins may be identified through database searching via computer procedures as described herein. “Peptide” or “protein” may also be used throughout the document as specific, but non-limiting exemplars of biomolecules, such as in describing “Peptide Detection.”

By “query” is meant a selection of a particular action, generally to answer a question. In one example of a query, ions may be subjected to MS/MS based on a list that is stored with the software. Alternatively, one can manually select ions to be subjected to MS/MS. This manual selection is also a query.

By “scan” is meant a mass spectrum from a single sample. Each fraction of a separation that is measured results in a scan. If a biomolecule is located in more than one fraction analyzed, then the mass spectrum for the biomolecule is present in more than one scan.

By an “underivatized” biomolecule or fragment thereof is meant a biomolecule or fragment thereof that has not been chemically altered from its natural state. Derivitization may occur during non-natural synthesis or during later handling or processing of a biomolecule or fragment thereof.

By an “unlabeled” biomolecule or fragment thereof is meant a biomolecule or fragment thereof that has not been derivatized with an exogenous label (e.g., an isotopic label or radiolabel) that causes the biomolecule or fragment thereof to have different physicochemical properties to naturally synthesized biomolecules

The invention, Constellation Mapping, is a bioinformatics tool that can be used, for example, to align peptides detected within a pair of mass spectrometric injections. The injection pair can be either LC-MS to LC-MS; LC-MS to LC-MS-MS; or LC-MS-MS to LC-MS-MS. The peptide alignment is generated utilizing pattern matching and iterative refinement techniques.

The methods and systems of the invention provide a number of significant advantages. For example, the methods and systems combine mass spectrometry and data analysis in a way that allows the direct comparison of the abundance of biomolecules without relying on derivatizing or labeling of the biological sample. The invention is robust to global retention time shifts such as liquid chromatography (LC) column offsets and robust to local retention time shifts, adjusting data from injections to render them comparable, and generating a nonlinear retention time transformation function that can be used for the prediction of biomolecule elution from one LC system to another. The information from the entire mass spectrum can also be used to determine expression levels and to correct for retention time variation, without a need for reference injections. Typically, without using Constellation Mapping, a large amount of information present in the mass spectra would be discarded, and only a subset, such as intensities of specific ions, or the sequence of specific peptides, or a list of peptide masses would be analyzed. Constellation Mapping determines an intensity normalization between the pair of injections based on common biomolecules, useful for comparing the abundance of biomolecules, however biomolecule alignment and retention time correction are intensity independent, and so, can be applied to injections that are significantly different. CM permits the detection of shared biomolecules between injections as well as identifying biomolecules unique to the injections. And, the use of automation greatly reduces the time necessary for analysis, as Constellation Mapping is extremely fast thereby allowing the thousands of peptide alignments, such as is needed in large-scale proteomic studies.

Other features and advantages of the invention will be apparent from the following drawings and detailed description, and from the claims.

### BRIEF DESCRIPTION OF THE DRAWINGS

The patent or application file contains at least one drawing executed in color. Copies of this patent or patent application publication with color drawings(s) will be provided by the Office upon request and payment of the necessary fee.

FIG. 1 illustrates an exemplary embodiment of a computer system of this invention.

FIG. 2 shows an example of the constellation mapping method, in this case to produce and align peptide maps. 1) Sample 1 is analyzed by mass spectrometry by acquiring LC/MS data, in this illustrated case, on a band of a 1D gel. The LC/MS data undergoes data format conversion, and centroiding and noise reduction, which generally reduces the file size. This results in an isotope map, which is used in peptide detection for the acquisition of data (such as m/z, retention time, charge, intensity, and area), and in turn results in a peptide map. 2) The procedure followed for 1) is followed for a second sample, illustrated in this case for the band in a 1D gel of sample 2 corresponding to the band analyzed for sample 1. The peptide maps of 1 and 2 are aligned and the peptides exhibiting differential abundance are determined. LC/MS-MS data can then be acquired on the differentially abundant peptides (targeted LC/MS-MS), followed by identification of the peptide and/or protein. Acquisition of 1, 2, 3, and 4 is interleaved on the same mass spectrometer, using the same column to minimize sample variability.

FIG. 3 shows an exemplary Noise Reduction Module Flow Chart.

FIG. 4 shows an exemplary Isotope Map. Intensity is depicted by shading, with a lighter shade indicating higher intensity. The m/z and rt dimensions appear on the horizontal and vertical axes, respectively.

FIG. 5 shows exemplary Isotope maps generated by nLC-MS analysis. The complete injection profile shown on the left shows several thousand peptide ions, separated by mass/charge ratio (vertical axis) and retention time in minutes (horizontal axis). An enlarged region is shown on the upper right, similar to that seen in FIG. 6, and a single peptide ion isotopic profile is shown on the lower right, similar to that shown in FIG. 7.

FIG. 6 shows an exemplary Isotope Map at medium resolution (x and y axes interchanged relative to FIG. 5).

FIG. 7 shows an exemplary Isotope Map at high resolution (x and y axes interchanged relative to FIG. 5). Note the striated pattern produced by groups of isotopes.

FIG. 8 shows an exemplary Peptide Detection Module Flow Chart.

FIG. 9 shows an example of an Isotope Map converted to a Peptide Map. The complex isotope map shown in the upper panel is converted to a lower complexity peptide map shown in the lower panel. Each peptide isotopic profile is replaced with a single point consisting of the mass, charge, retention time and abundance of that peptide. The symbols represent the detection of charge +1, +2, +3 and +4 (circle, cross, triangle, square) peptides.

FIG. 10 illustrates Peptide Detection. The corresponding peptide map (see FIG. 9 for example) is overlaid on the isotope map from which it was derived to illustrate the “centering” of peptides.

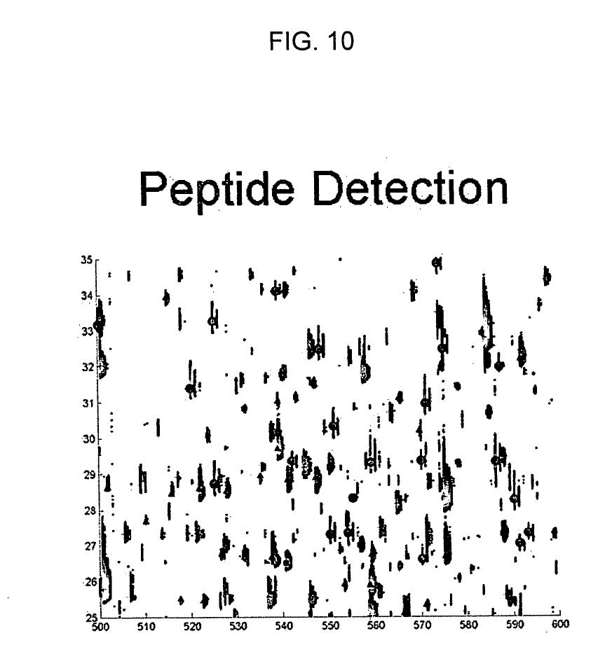

FIG. 11 also illustrates Peptide Detection. The corresponding peptide map (see FIG. 9 for example) is overlaid on the isotope map from which it was derived to illustrate the “centering” of peptides.

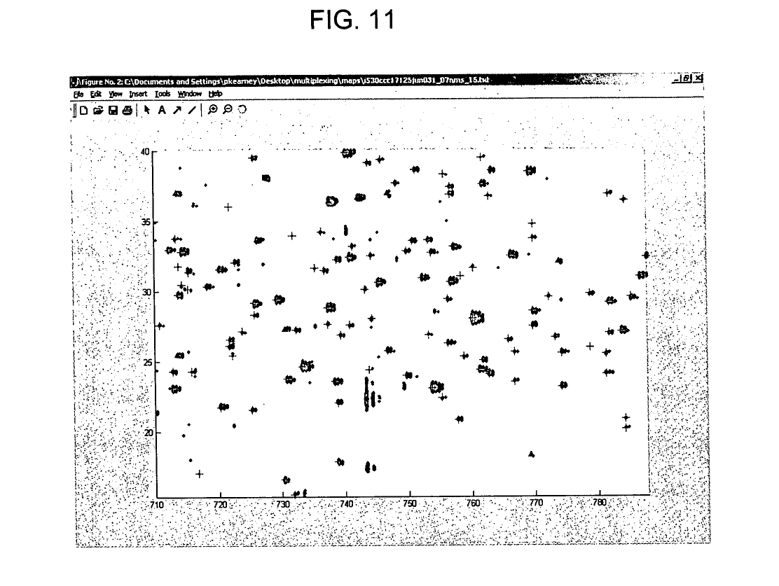

FIG. 12 also shows an exemplary Peptide Map. The different shapes (triangle, circle, square, plus sign) designate the charge state of the ion.

FIG. 13 shows an exemplary Peptide Map Alignment Module Flow Chart.

FIG. 14 shows two representative peptide maps that might undergo Peptide Map Alignment.

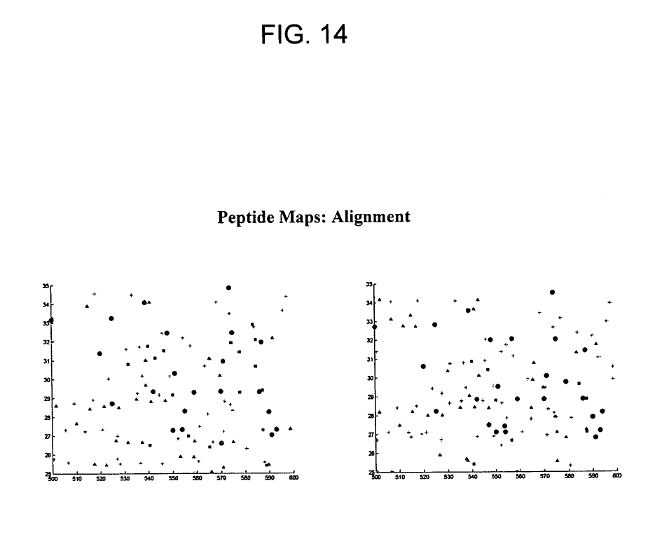

FIG. 15 shows a representative aligned peptide map (map A) at 1/40^{th} the area for a complete scan for comparison with FIG. 16.

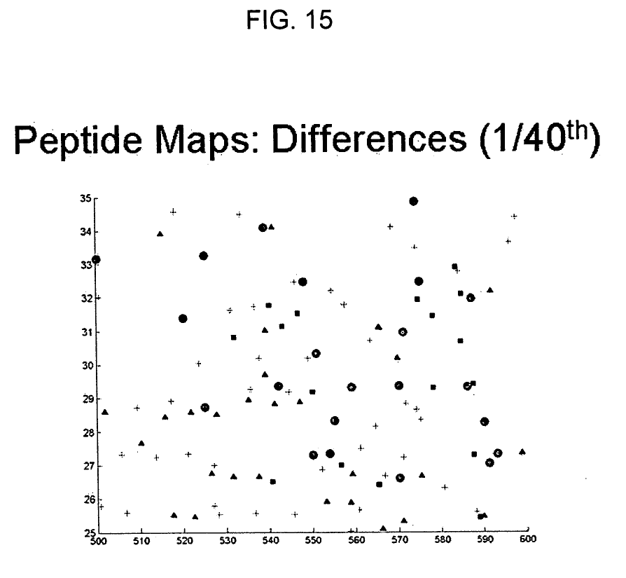

FIG. 16 shows a representation at 1/40^{th} the area for a complete scan of visualized differences between aligned peptide maps (A and B), in this case unmatched peptides from map B, shown circled. Compare with FIG. 15, which would represent map A of the two aligned maps.

FIG. 17 Retention Time Transformation Function. An example of the dynamic offset routine allows for the matching of peptides in two different LC-MS spectra, independent of the variability introduced by different pumps, different columns, or pump rate fluctuations. The blue line is the learned retention time correction function required for matching peptides reliably. Circles near the line are matched between samples. Circles far off the line are not matched and therefore unique to the first sample.

FIG. 18 illustrates alignment of a map from LC-MS with a map from LC-MS-MS. At upper right is shown a fragmentation spectrum from LC-MS-MS, and part of corresponding peptide map is shown on lower right. At lower left is part of the peptide map from the LC-MS injection.

FIG. 19 illustrates the distribution of the coefficient of variation over 15 injections using Constellation Mapping.

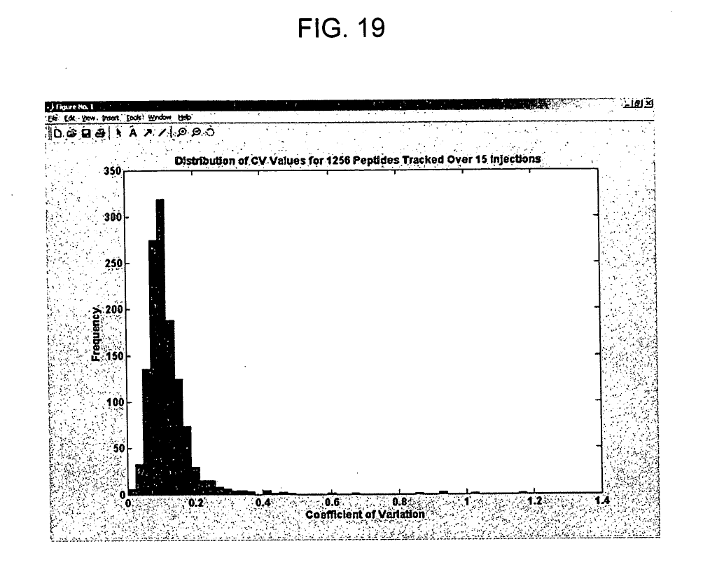

FIG. 20 illustrates an intensity scatter plot comparing the intensities of aligned peptides from one injection to another.

FIG. 21 illustrates calculating peptide abundance from intensity or volume.

### DETAILED DESCRIPTION OF THE INVENTION

The invention features methods and software for generating retention time offsets and comparing the abundance of one or more biomolecules, qualitatively or quantitatively, or both, between two or more samples. In one application, the methods and systems of the invention are used to compare a large number of peptides present in two or more samples in order, for example, to determine variations in relative expression levels or to identify peptides for which ratios of relative expression are above or below pre-set values. Statistical analysis of expression profiles can then be used to identify peptide markers, such as for disease diagnostics and drug discovery.

Biological Samples

Using the methods of the invention, an expression profile of one or more biomolecules can be monitored in a biological sample. Exemplary biomolecules useful in the methods of the invention include any molecule that is present in a biological sample, e.g., peptides, polypeptides, proteins, post-translationally modified peptides (e.g., glycosylated, phosphorylated, or acylated peptides), oligosaccharides and polysaccharides, lipids, nucleic acids, and metabolites. Virtually any biological sample is useful in the methods of the invention, including, without limitation, any solid or fluid sample obtained from, excreted by, or secreted by any living organism, including single-celled micro-organisms (such as bacteria and yeasts) and multicellular organisms (such as plants and animals, for instance a vertebrate or a mammal, and in particular a healthy or apparently healthy human subject or a human patient affected by a condition or disease to be diagnosed or investigated). A biological sample may be a biological fluid obtained from any location (such as blood, plasma, serum, urine, bile, cerebrospinal fluid, aqueous or vitreous humor, or any bodily secretion), an exudate (such as fluid obtained from an abscess or any other site of infection or inflammation), or fluid obtained from a joint (such as a normal joint or a joint affected by disease such as rheumatoid arthritis). Alternatively, a biological sample can be obtained from any organ or tissue (including a biopsy or autopsy specimen) or may comprise cells (whether primary cells or cultured cells) or medium conditioned by any cell, tissue, or organ. If desired, the biological sample is subjected to preliminary processing, including preliminary separation techniques. For example, cells or tissues can be extracted and subjected to subcellular fractionation for separate analysis of biomolecules in distinct subcellular fractions, e.g., proteins or drugs found in different parts of the cell. Such exemplary fractionation methods are described in De Duve ((1965) J. Theor. Biol. 6:33-59).

When analyzing proteins, a biological sample, if desired, is purified to reduce the amount of any non-peptidic materials present. Moreover, if desired, protein-containing samples are cleaved to produce smaller peptides for analysis. Cleavage of the peptides is generally accomplished enzymatically, e.g., by digestion with trypsin, elastase, or chymotrypsin, or chemically, e.g., by cyanogen bromide. The cleavage at specific locations in a protein can allow the prediction of the masses of the smaller peptides produced if the sequences of these peptides are known. All samples that are to be compared typically are treated in the same manner.

A reference sample, if desired, can also be included when performing the methods described herein. This reference sample typically includes known amounts of biomolecules or may be derived from a known source, e.g., a non-diseased tissue. The reference sample may be synthesized from known biomolecules. Additionally, unknown samples may be compared to the reference sample to determine a relative abundance. Reference samples may also be combined with other samples to act as internal standards where appropriate.

Separation of Biomolecules

A wide variety of techniques for separating any of the aforementioned biomolecules are well known to those skilled in the art (see, for example, Laemmli Nature 1970, 227:680-685; Washburn et al., Nat. Biotechnol. 2001, 19:242-7; Schagger et al., Anal. Biochem. 1991, 199:223-31) and may be employed according to the present invention.

In one application, the methods of the invention are used to study complex mixtures of proteins. By way of example, mixtures of proteins may be separated on the basis of isoelectric point (e.g., by chromatofocusing or isoelectric focusing) and/or of electrophoretic mobility (e.g., by non-denaturing electrophoresis or by electrophoresis in the presence of a denaturing agent such as urea or sodium dodecyl sulfate (SDS), with or without prior exposure to a reducing agent such as 2-mercaptoethanol or dithiothreitol), by chromatography, including LC, FPLC, and/or HPLC, on any suitable matrix (e.g., gel filtration chromatography, ion exchange chromatography, reverse phase chromatography, or affinity chromatography, for instance with an immobilized antibody or lectin or immunoglobins immobilized on magnetic beads), and/or by centrifugation (e.g., isopycnic centrifugation or velocity centrifugation).

In some cases, two different peptides may have the same mass within the resolution of a mass spectrometer, rendering determination of abundances for those two peptides difficult. Separating the peptides before analysis by mass spectrometry allows for the resolution of the abundances of two peptides with the same mass. Although many spectra for the fractions of the separation may then be obtained, these spectra typically have a reduced number of ion peaks from the peptides, which simplifies the analysis of a given spectrum.

In one embodiment, a mixture of proteins is separated by ID gel electrophoresis according to methods known in the art. The lane containing the separated proteins is excised from the gel and divided into fractions. The proteins are then digested enzymatically. The peptides produced in each fraction are then analyzed by mass spectrometry. For example, proteins from plasma membrane fractions from normal and tumour tissues are solubilized and fractionated by iD SDS polyacrylamide gel electrophoresis (PAGE). Gels are cut into 24 equal bands and each band is digested by trypsin to obtain peptides for analysis by nano-liquid chromatography-mass spectrometry (LC-MS). Each peptide fraction is injected onto a nano-liquid chromatography C₁₈ column, coupled by electrospray to a QTOF (quadrapole time of flight) mass spectrometer.

In another embodiment, peptides are separated by 2D gel electrophoresis according to methods known in the art. The proteins are then digested enzymatically, and the digested peptides produced in each fraction are then excised and analyzed by mass spectrometry. In still another embodiment peptides are separated by liquid chromatography (LC) by methods known in the art, including, but not limited to, multidimensional LC. LC fractions may be collected and analyzed or the effluent may be coupled directly into a mass spectrometer for real-time analysis. LC may also be used to separate further the fractions obtained by gel electrophoresis. Recording the retention time (RT) of a peptide in LC can enable the identification of that peptide in multiple fractions. This identification is typically useful for obtaining an accurate abundance. In any of the above embodiments, a given peptide may be present in more than one fraction depending on how the fractions were obtained.

Mass Spectrometry

Exemplary methods for analyzing biomolecules using mass spectrometry techniques are well known in the art (see Godovac-Zimmermann et al. (2001) Mass Spectrom. Rev. 20:1-57 (PMID: 10344271); Gygi et al. (2000) Proc. Natl. Acad. Sci. U.S.A. 97:9390-9395 (PMID: 10920198)).

In applications involving peptides, the peptides are ionized, e.g., by electrospray ionization, before entering the mass spectrometer, and different types of mass spectra, if desired, are then obtained. The exact type of mass spectrometer is not critical to the methods disclosed herein. For example, in a survey scan, mass spectra of the charged peptides in a sample are recorded. Furthermore, the amino acid sequences of one or more peptides may be determined by a suitable mass spectrometry technique, such as matrix-assisted laser desorption/ionization combined with time-of-flight mass analysis (MALDI-TOF MS), electrospray ionization mass spectrometry (ESI MS), or tandem mass spectrometry (MS/MS). In a MS/MS scan, specific ions detected in the survey scan are selected to enter a collision chamber. The ability to define the ions for MS/MS allows data to be acquired for specific precursors, while potentially excluding other precursors. The ions may be defined by a predetermined list or by a query. Lists may be inclusion lists (i.e., ions on the list are subjected to MS/MS) or exclusion (i.e., ions on the list are not subjected to MS/MS). The series of fragments that is generated in the collision chamber is then itself analyzed by mass spectrometry, and the resulting spectrum is recorded and may, for example, be used to identify the amino acid sequence of a particular peptide processed in this manner. This sequence, together with other information such as the peptide mass, may then be used, e.g., to identify a protein. The ions subjected to MS/MS cycles may be user defined or determined automatically by the spectrometer.

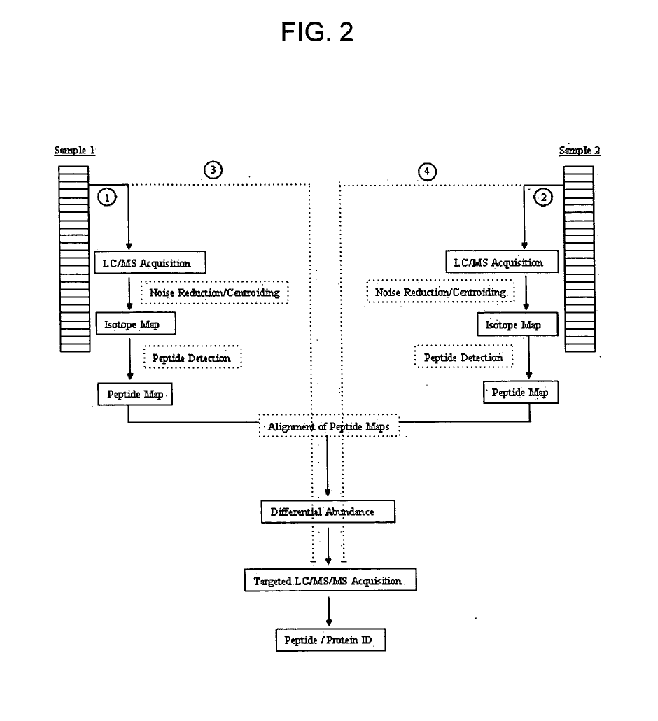

In a preferred embodiment, variability between samples to be compared is minimized by interleaving. For example, mass spectrometry is performed on band 1 of sample 1, then band 1 of sample 2 on the same column of the same machine, MS-MS would then be performed on band 1 of sample 1, then band 1 of sample 2, and then the procedure could be performed for band 2 of each sample (see FIG. 2). Also in a preferred embodiment, Constellation Mapping is run in real time, to minimize variability by allowing the selection of differentially abundant peptides for MS-MS so that a pattern of interleaving can be followed.

Constellation Mapping (CM)

Software to analyze mass spectra is typically used to identify the biomolecule from which an ion was derived. Comparing LC-MS scans, however, can be extremely difficult given local non-linear variation in retention times. As is described herein, an automated approach allows the processing of mass spectra recorded for two or more samples so that a comprehensive comparison of the biomolecules in the samples can be achieved, and, differentially abundant biomolecules can be identified and selected for a subsequent round of MS-MS, potentially including those performed in real time,.

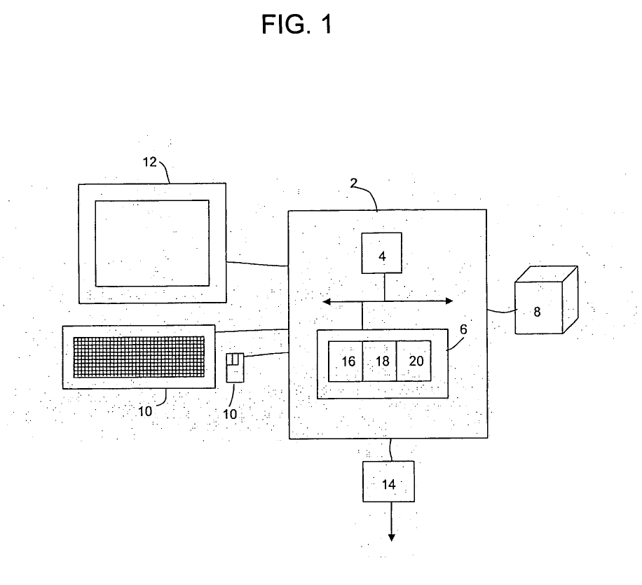

The methods described herein are implemented using virtually any computer system and according to the following exemplary programs. FIG. 1 shows an exemplary computer system. Computer system 2 includes internal and external components. The internal components include a processor 4 coupled to a memory 6. The external components include a mass-storage device 8, e.g., a hard disk drive, user input devices 10, e.g., a keyboard and a mouse, a display 12, e.g., a monitor, and usually, a network link 14 capable of connecting the computer system to other computers to allow sharing of data and processing tasks. Programs are loaded into the memory 6 of this system 2 during operation. These programs include an operating system 16, e.g., Microsoft Windows, which manages the computer system, software 18 that encodes common languages and functions to assist programs that implement the methods of this invention, and software 20 that encodes the methods of the invention in a procedural language or symbolic package. Languages that can be used to program the methods include, without limitation, Visual C/C⁺⁺ from Microsoft. In preferred applications, the methods of the invention are programmed in mathematical software packages that allow symbolic entry of equations and high-level specification of processing, including procedures used in the execution of the programs, thereby freeing a user of the need to program procedurally individual equations or procedures. An exemplary mathematical software package useful for this purpose is Matlab from Mathworks (Natick, Mass.). Using the Matlab software, one can also apply the Parallel Virtual Machine (PVM) module and Message Passing Interface (MPI), which supports processing on multiple processors. This implementation of PVM and MPI with the methods herein is accomplished using methods known in the art. Alternatively, the software or a portion thereof is encoded in dedicated circuitry by methods known in the art. CM offers significantly increased speed of analysis compared to performing the methods herein manually.

In one application, the invention features computer implemented modules for studying proteins. Such modules are described here as exemplars of the methods of the invention. Other biomolecules may be studied using similar modules. CM, if desired, can be run simultaneously in a multiprocessing environment to reduce the time required for analysis. The multiprocessing environment, for example, includes a cluster of systems (e.g., Linux-based PCs) or servers with multiple processors (e.g., from Sun Microsystems), and the methods herein are implemented onto such distributed networks using methods known in the art (see Taylor et al. (1997) Journal of Parallel and Distributed Computing 45:166-175).

A flowchart for an exemplary CM is shown in FIG. 2. Solid rectangles represent processing components of a CM, dashed rectangles represent processing components that are not within CM and entries without a rectangle are data files. Each component is described in detail below, exemplified as processing modules. This flowchart is presented for the purpose of illustrating, not limiting, the methods of the invention.

Noise Reduction and Centroiding

In the analysis of a biological sample by a mass spectrometer, the instrument records the different ions in the sample. The values measured in each scan are the m/z (mass/charge ratio), and the intensity or frequency of the ions (which also have retention time values from LC). The high sensitivity of the instrument results in the raw data generated in MS survey scans being plagued with a great percentage of background noise, which presents challenges in interpretation of the data. It is difficult to differentiate between weak signals and noise, because of the variable intensity of noise. And, the size of the raw data with noise makes downstream processing inefficient and impractical in terms of time and computing power, because of the complexity of analysis. However, limitations in sensitivity also increase this complexity by spreading the ion counts for a single biomolecule (different ions of the same chemical composition) across a range of m/z values, because of the least count of the mass spectrometer. For example, five molecules of mass 900 with a charge of 2 are observed by the mass spectrometer. The “real” m/z is the “ideal” m/z, i.e. 900/2=450, but the mass spectrometer measurements are a sampling of the “real” m/z—the mass spectrometer won't read all the peptides as being exactly 450.000000 in m/z, but will differ from the real value by, at most, the least count of the instrument, and may read in five different m/z values (e.g. 449.93, 450.01, 450.06, 450.0, 449.99), which might be interpreted as five peptides (or noise) of intensity 1, however, they actually represent 1 peptide with an intensity of 5. A noise reduction module can thus greatly enhance accuracy, sensitivity, and speed, and produce isotope maps, which provide a data source for a Peptide Detection Module.

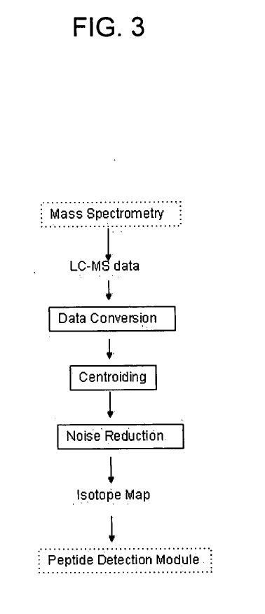

FIG. 3 is a flowchart detailing the components of a Noise Reduction Module (NRM). Solid rectangles represent processing components of an NRM, dashed rectangles represent processing components that are not within an NRM and entries without a rectangle are data files. Each component is described in detail below. This flowchart is presented for the purpose of illustrating, not limiting, the methods of the invention.

Data Format Conversion

Raw mass spectrometry data files typically consist of MS scans or a series of survey scans and MS/MS cycles for each fraction of a separation. Each mass spectrum corresponds, e.g., to an elution time period for LC or to a fraction for gel electrophoresis, or both. Each survey scan records the number of ions of each m/z value detected by the mass spectrometer. Raw mass spectrometry data files may be generated by various publicly available software packages including, without limitation, MassLynx from Micromass (Beverly, Mass.). To integrate CM with, e.g., MassLynx, software in MassLynx converts the data from the mass spectrometer, for example, (e.g. Masslynx format.raw) into an ASCII or NetCDF format. Other software packages for obtaining mass spectrometry data have similar conversion software. Alternatively, software for data conversion is written using methods known in the art and included in the module. Optionally, data conversion, may also include merger of multiple files. File merger may also include merger of elements of the files, such as the abundances of particular precursors.

Centroiding

Ions of a species (ion count measurements of a particular biomolecule and of the same charge state, but differing m/z values) are recorded by a mass spectrometer as a distribution around the “real” m/z value of the biomolecule (see example in Noise Reduction and Centroiding above). Centroiding is performed to consolidate the range of values (ions of a species) the mass spectrometer produces for biomolecules. Centroiding algorithms are commonly known in the art. The data acquired for each biomolecule of a particular charge state could thus be represented by a single m/z value and an associated ion count. For example, a centroiding algorithm could calculate a single “real” m/z for the five ions in the above mentioned example that is an average of the five m/z values and sum the intensities (e.g. m/z=449.998, intensity=5) to represent the ions of the species, and this could then be used to replace the distribution of ions. Centroided data can in turn be integrated across scans for ions of species.

Noise Removal

Centroided data is inspected and local noise removed. In one embodiment, noise removal is a simple deletion of all low intensity ion counts, or ion counts below a certain threshold. A threshold of ion intensity may be defined to differentiate signal from peptide ions from those of noise. This threshold can be estimated for all scans by using methods known in the arts, such methods include, without limitation, the method of Maximum Entropy.

Isotope Map Generation

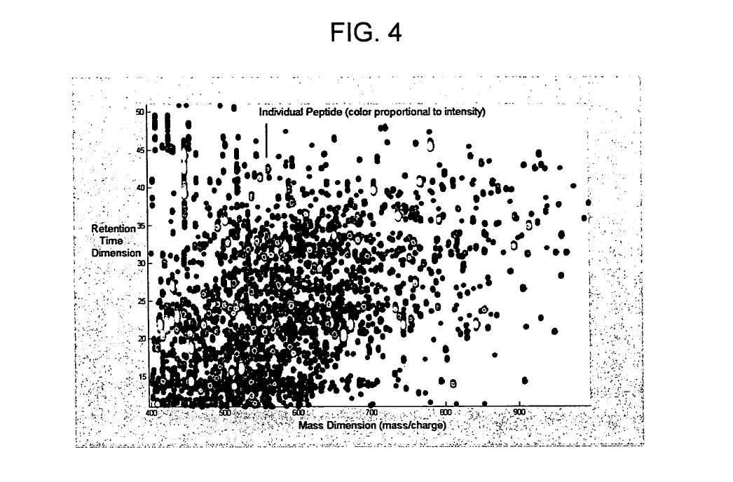

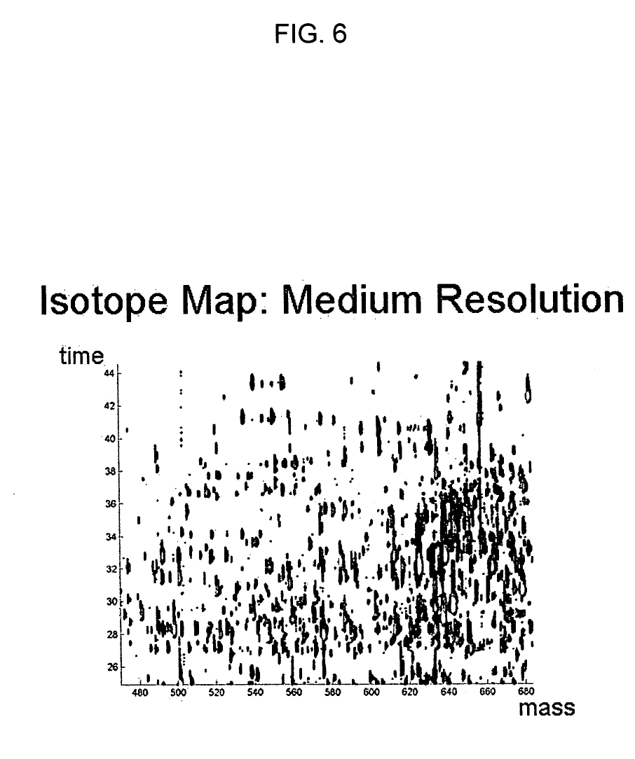

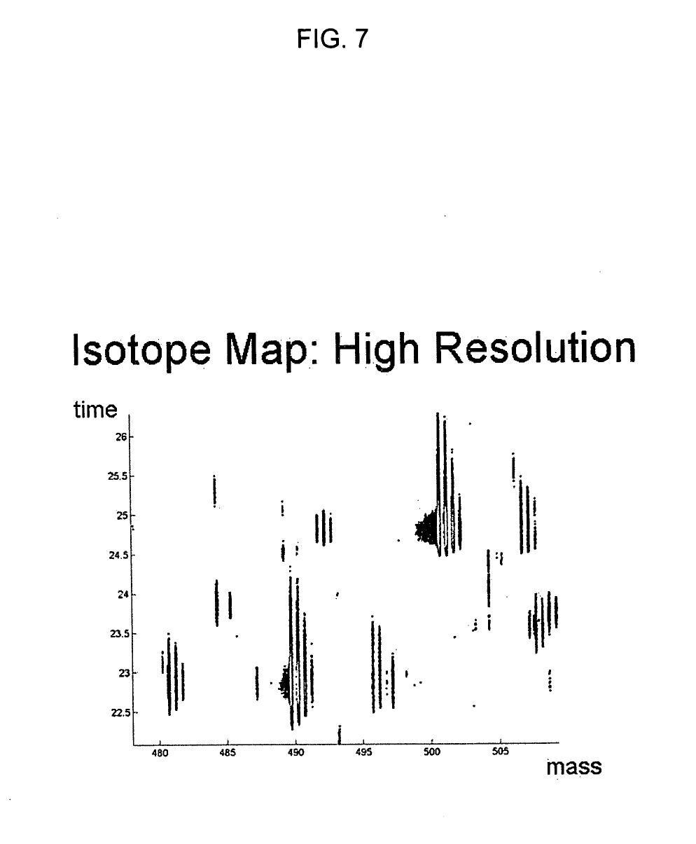

Centroided and noise reduced data can be processed to produce an isotope map for LC-MS (or LC-MS-MS) data, comprising triples of mass-to-charge ratio (m/z), retention time (rt), and intensity for the biomolecules in the sample. A biomolecule may thus be represented within an isotope map as a series of isotopes spaced at predictable mass differences depending on the charge of the biomolecule (e.g. a peptide). Generally such a map is made for the data from an injection. In one embodiment the map is generated as a text file. In a related embodiment, the text file may be visualized (see for example, FIGS. 4, 5, 6, and 7).

Peptide Detection

An isotope map represents peptides by their mass, retention time, charge state and intensity (see FIG. 4). The mass, retention time and intensity of a peptide corresponds to the most intense peak in the first isotope of a peptide's isotopes in the isotope map. This is called the peptide's “center.” The detection of peptide centers in isotopes is based on the following properties:

A peptide's isotopes are distributed across retention time, and so, can be distinguished from random noise.

The spacing and intensity of a peptide's isotopes can be modeled, and so, recognized within an isotope map.

There are four steps in peptide detection: determining local mass maxima, determining local retention time maxima, eliminating local maxima based on isotope density, and peak charge determination. These steps can be followed by the production of a peptide map.

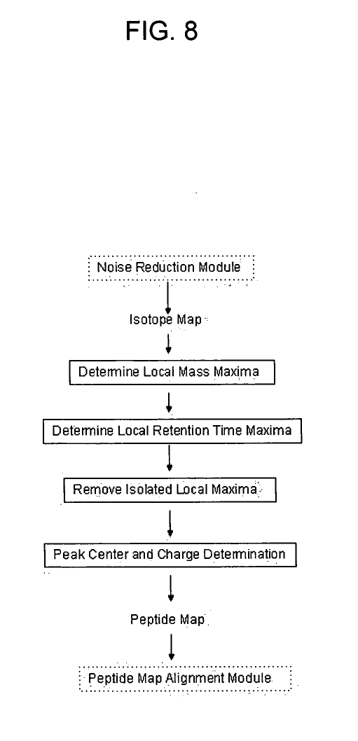

FIG. 8 is a flowchart detailing the components of a Peptide Detection Module (PDM). Solid rectangles represent processing components of a PDM, dashed rectangles represent processing components that are not within PDM and entries without a rectangle are data files. Each component is described in detail below. This flowchart is presented for the purpose of illustrating, not limiting, the methods of the invention.

### Local Mass Maxima

Within an isotope map, all local maxima within a given scan (i.e. retention time) are found. A local maximum is defined by a mass window typically set to be the width of an isotope. This reduces the amount of data significantly since most data points are not local maxima.

### Local Retention Time Maxima

Within an isotope map, every peak that is a local maximum within a mass and retention time window centered at the peak, is found. This step is performed only on those peaks determined to be local mass maxima in the previous steps for efficiency. The mass and retention time window is typically defined to enclose an entire isotope. As above, the amount of data is significantly reduced by this step.

### Isotope Density

To remove isolated local maxima, only those local retention time maxima are kept that have a significant number of local mass maxima both above and below. This is a property that isotopes will have but noise will typically not have.

### Peak Centers and Charge Detection

Among the remaining peaks, those which are peptide centers are detected and the charge determined. For each peak, the hypothesis that it is a peptide center of a charge k peptide is evaluated. This is achieved by checking for the existence of isotope centers of putative 2^{nd}, 3^{rd} and/or 4^{th} isotopes. The intensities of these isotopes are compared to the intensity of the putative peptide center for consistency. Methods for charge determination and isotope detection could include or be similar to those found in U.S. Utility patent application Ser. No. 10/293,076 “Mass Intensity Profiling System and Uses Thereof”, which is hereby incorporated by reference.

### Peptide Map Generation

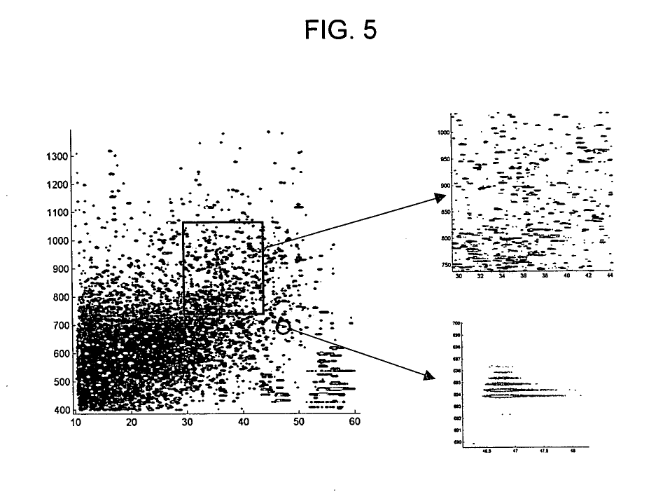

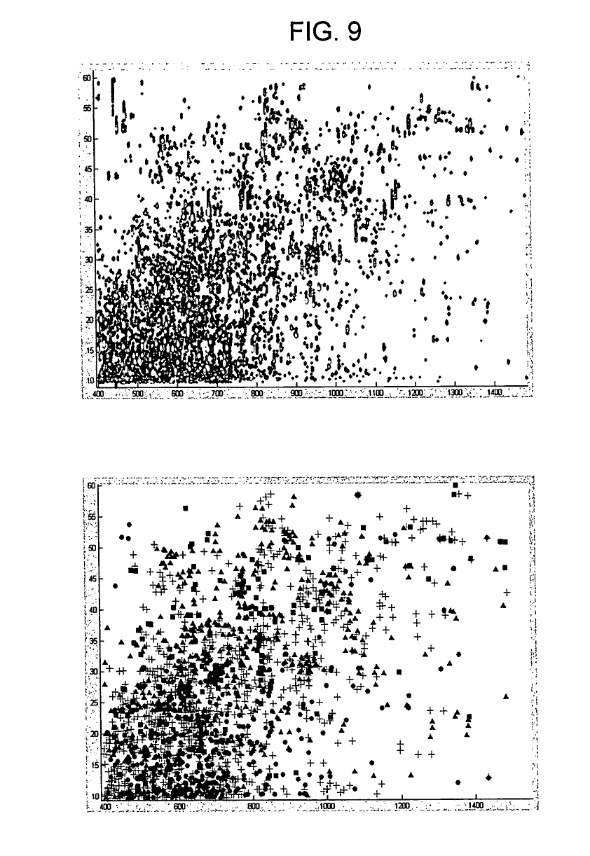

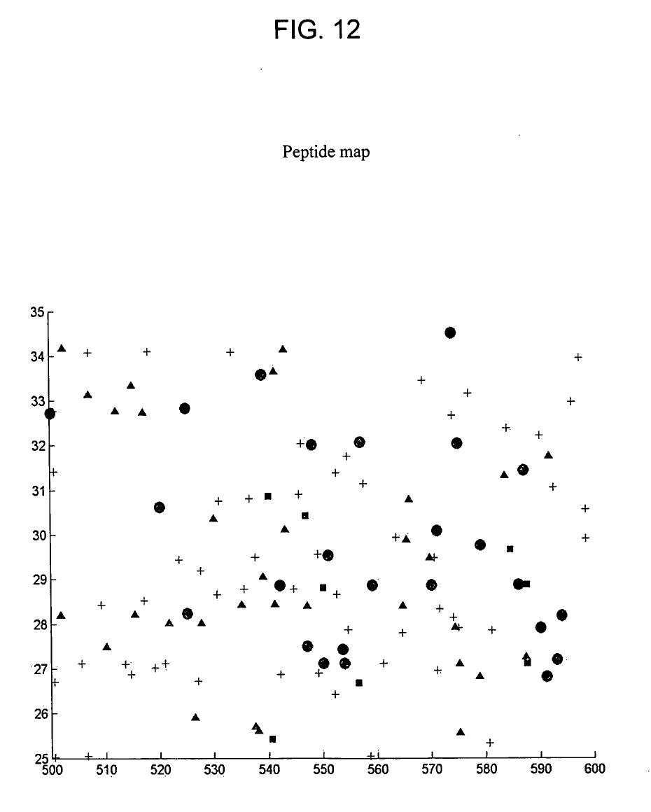

An isotope map from biological sample, such as tumor tissue, can typically have several thousand peptide ions visible, separated by retention time and a mass/charge ratio. While the image is complex, individual peptides can be readily detected. The images are too data intensive, however, to make comparisons across patients a rapid and reliable process. For this reason, each isotope map is converted to a peptide map, as shown in FIG. 9, 10, 11, and 12. Each complex peptide isotope signature, such as shown in FIG. 5, lower right, is replaced with a single point, represented by the mass, charge, retention time, and abundance of that peptide. Thus, a peptide map may be generated from the processed isotope map data (see FIG. 4), with each peptide (or biomolecule) comprising a quartet of mass-to-charge ratio (m/z), retention time (rt), charge (ch), and intensity. This greatly simplified data set allows for a rapid and accurate comparison across many samples. In one embodiment the map is generated as a text file. In a related embodiment, the text file may be visualized (see for example, FIG. 12).

Alignment of Peptide Maps

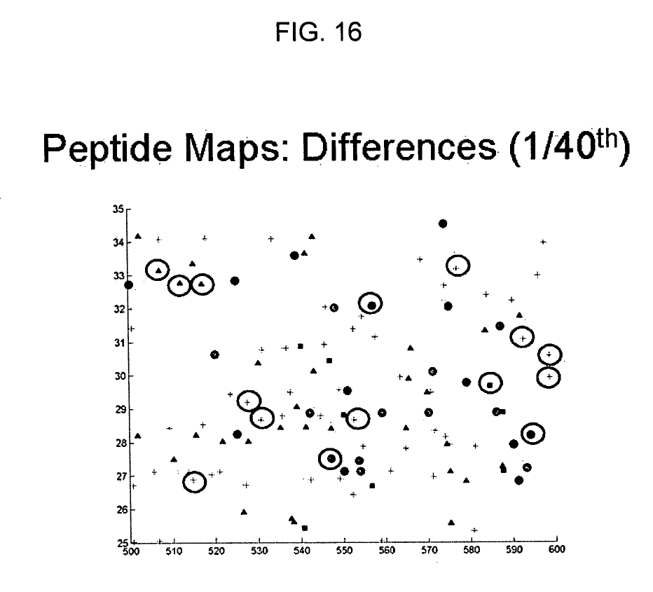

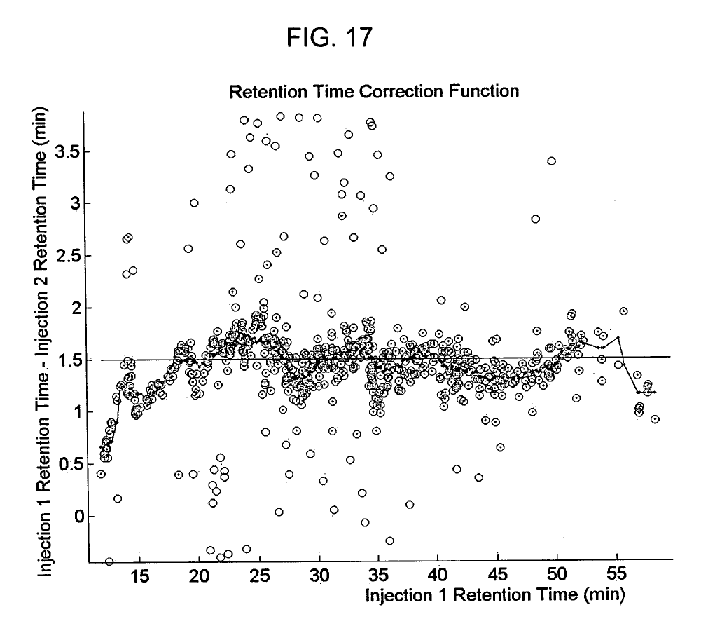

Given two peptide (or biomolecule) maps A and B, in order to determine differentially abundant peptides, peptides in A must be matched to peptides in B (see FIG. 16). Accurate matching of peptides between samples is critical to a successful analysis. Due to limitations in reproducibility in the flow of capillary nano-liquid chromatography pumps, the retention time for a given peptide can vary by 2% from run to run, particularly if comparing across different liquid chromatography columns or pumps. This variability can also differ across the run, resulting in an offset of up to 2 minutes in either direction. To deal with this, a dynamic offset correction has been devised to match the retention time when comparing two or more samples. The offset is based on pattern matching at each time point, resulting in the ability to accommodate even highly erratic behavior as shown in FIG. 17. Reference injections are not needed: two LC-MS injections can be directly compared. RT correction is also independent of intensity values, so under conditions where peptide content and intensities are expected to vary, still performs well. Non-identical samples with varied peptide content can be profiled and differences detected. Also identified in this process are those peptides which are unique to one or the other sample, shown as points off the line of correlation (FIG. 17).

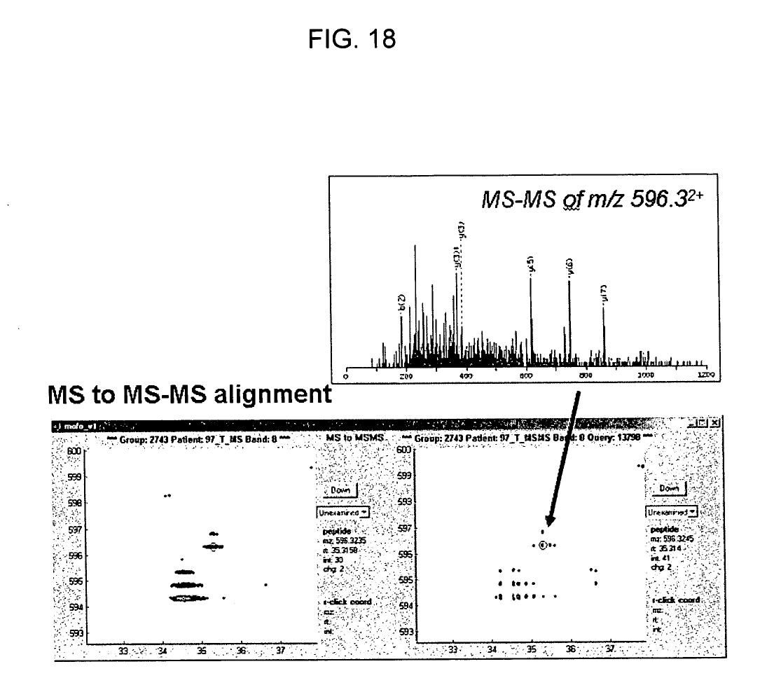

In sum, for a pair of injections (LC-MS to LC-MS; LC-MS to LC-MS-MS (see, for example, FIG. 18); or LC-MS-MS to LC-MS-MS) peptide alignment can be readily used to generate information such as:

The column retention offset between the pair of injections being compared.

A retention time transformation function from injection 1 to injection 2.

A linear intensity normalization function from injection 1 to injection 2.

The list of shared and unique peptides for injection 1 and injection 2.

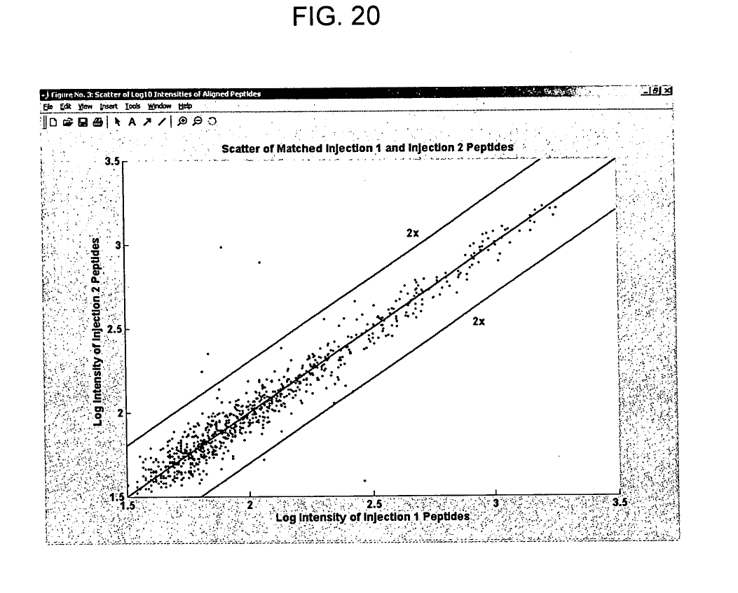

For example, FIG. 17 depicts the predicted column offset (solid black line) and the retention time transformation function for a pair of injections. FIG. 20 depicts an intensity scatter plot that compares the intensities of aligned peptides from injection 1 to injection 2.

Algorithm

Again, due to variations in mass and retention time, the alignment of peptide maps is not straightforward. In particular, variation in retention time can compress and/or expand on a local basis, and so, linear alignment schemes can yield poor results. Since mass variability is low relative to retention time variability, the challenge of matching peptides is mainly to find a function that maps the retention times of peptides in A to peptides in B.

The algorithm has two main steps:

1. The column offset between the pair of injections is predicted.

2. A local retention time transformation between the pair of injections is predicted.

These steps can be further subdivided, and the process of peptide map alignment can be described as five steps: determining peptide neighbors, retention time clustering, best adjustment, iteration and optimization, and application of adjustment.

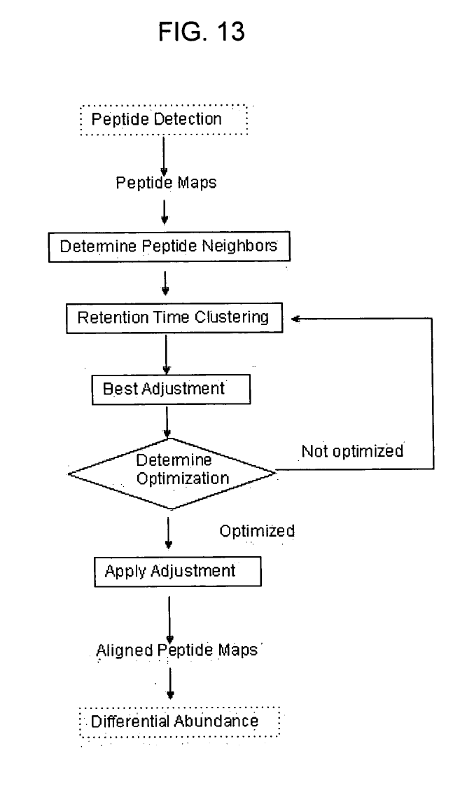

FIG. 13 is a flowchart detailing the components of a Peptide Map Alignment Module (PMAM). Solid rectangles represent processing components of a PMAM, dashed rectangles represent processing components that are not within PMAM and entries without a rectangle are data files. This flowchart is presented for the purpose of illustrating, not limiting, the methods of the invention. Each component (the five steps the algorithm) is described in detail below, plus an optional initial step.

### [Optional] Removal of Low Information Molecules

All peptides may be used to correct for rt variation. However, optionally, low information peptides such as singly charged or low intensity peptides can be omitted in order to derive a high quality retention time transformation function. These peptides can be later reinstated before step 5 (application of adjustment) below.

### 1) Peptide Neighbors

Peptides are loosely aligned between injection by matching on m/z, rt and, optionally, charge: for each peptide p in A, define the neighbors of p in B to be all peptides in B of the same charge as p and within a predefined mass and retention time window of p. The mass and retention time window will depend on the variability of the system. The m/z matching tolerance is typically very precise (less than 0.10 Da). Matching on charge is exact, if it is employed. The rt matching tolerance is defined loosely depending on the application of the alignment but is typically less than 8 minutes. These matches are depicted as red in FIG. 17.

The steps below attempt to correctly match p to one of its neighbors in B.

### 2) Retention Time Clusters

The column offset is determined by analyzing the distribution of retention time offsets for all loosely matched peptides, such as by sorting the peptides in p from low to high retention time, randomly grouping peptides into clusters of peptides of similar retention time (i.e. within a predefined difference). These groupings are called retention time clusters. Since peptides within the clusters have similar retention time, the algorithm will attempt to adjust the retention time of all of these peptides by the same amount. Typically, the distribution mode is used to define the column offset but any measure of centrality can be used.

### 3) Best Adjustment

For each retention time cluster, the optimum retention time adjustment is determined. The constraint is that all peptides within the cluster can only be matched to one of its peptide neighbors in B and that the retention time adjustment is shared by all of the peptides within the cluster. Algorithmically, the optimum retention time adjustment can be determined by many approaches including integer programming. Typically, matched peptides within +/−2 minutes (or some other empirically determined value) of the column offset are kept for further analysis.

A median smoothing window is applied along retention time to obtain local retention time offset values. This results in the blue line depicted in FIG. 17.

### 4) Repeat and Optimize

Steps 2 and 3 are repeated k times and the optimal solution is kept. An optimal solution is one that minimizes the retention time adjustment over all retention time clusters.

### 5) Apply Adjustment

The optimal retention time adjustment is applied to all retention time clusters. If a peptide is within a predefined retention time threshold of one of its neighbors then they are matched. Typically, matched peptides within +/−0.5 minutes (or some other empirically determined value) of the median smoothed function are selected as the final matched peptides. Otherwise, the peptide remains unmatched and is considered to be unique to A or B. Intensity normalization is determined by linear regression on the matched peptides.

Differential Abundance

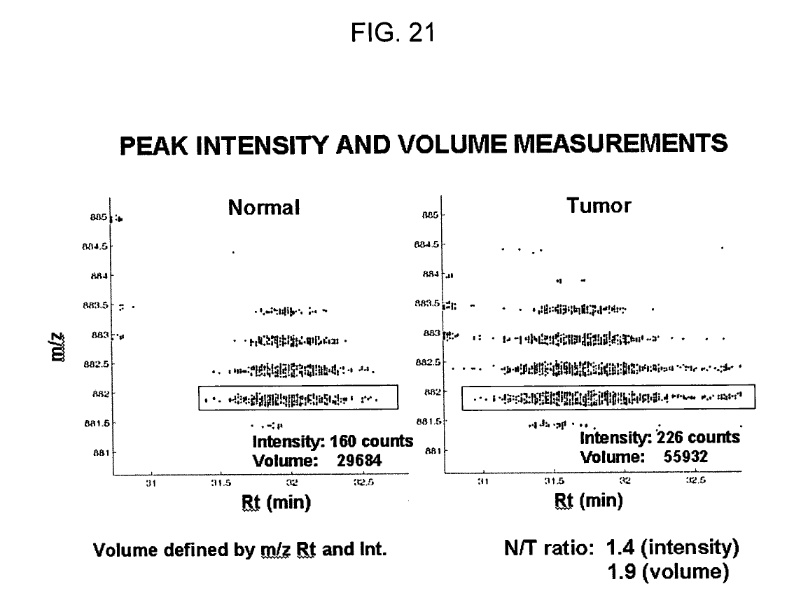

Peptide matching between samples can be followed by a determination of relative abundance for each peptide. Abundance is a function of the peak intensity or volume (as defined by m/z, rt, and intensity) as detected by the mass spectrometer (see FIG. 21), and its automated calculation can rely on methods such as those found in “Mass Intensity Profiling System and Uses Thereof” (U.S. Utility patent application Ser. No. 10/293,076). While each peptide has a unique ionization potential, making determination of absolute abundance difficult, the relative abundance of a peptide is directly related to its concentration in samples of similar complexity.

Matched peptides with differences in abundance greater than a given threshold, depending on the variability of the system, and, optionally, any unmatched peptides, may be selected for MS-MS (see FIG. 2). Differential abundance between peptide maps maybe visualized as exemplified in FIG. 16 and 20.

Peptide/Protein Identification

A large number of peptides in a sample can be identified through MS/MS analyses. An MS/MS cycle produces peptide sequence information on a selected peptide, which may then be used to search databases comprehensively. The raw mass spectrometry data can be submitted for compound, e.g., protein, identification using a tool such as Mascot from Matrix Science (London, United Kingdom), ProteinLynx Global Server from Micromass SEQUEST/TurboSEQUEST from Thermo Finnigan (San Jose, Calif.), or Sonar MS/MS from ProteoMetrics (New York, N.Y.). For example, a computer is used to search available databases for a matching amino acid sequence or for a nucleotide sequence, including an expressed sequence tag (EST), whose predicted amino acid sequence matches the experimentally determined amino acid sequence. Exemplary databases useful for this purpose include, without limitation, Genbank, EMBL, NCBI, MSDB, SWISS-PROT, TrEMBL, dbEST, Human Genome Sequence database, or a user-defined database. Sequence information on compounds in the databases that contain the selected peptide may then be used to produce a list of other peptides derived from that compound using a specified cleavage technique. This analysis generates a list of proteins that are likely to exist in the sample under analysis.

Integration Over Fractions or Bands. If samples analyzed by mass spectrometry are excised from 1D gels, the abundance of an observed peptide is typically integrated over neighboring bands since the peptide might appear in several bands. The same peptide in neighboring bands is identified, e.g., by mass, retention time, and MS/MS. If samples are analyzed by multidimensional LC (e.g., 2D), the abundance is typically integrated over salt fractions. Integration may be performed on data prior to map generation or from two or more maps.

Individual Peptide Abundance Statistical Analyses. The list of peptides masses, their abundances, and retention times are used for various analyses, such as protein identification by mass fingerprinting; protein identification, through defining peptides for a further round of MS/MS; protein identification that combines matching MS/MS and mass fingerprinting, which can increase the peptide coverage of a protein and assist in differentiating between similar proteins in a family or between splice variants and between polymorphisms; and determining low abundance peptides present in the raw mass spectrometry data, which may correspond to low abundance proteins in the sample being analyzed.

Expression Profiling

The methods of the present invention can be used to determine the relative abundance of a biomolecule or fragment thereof, e.g., proteins, in samples (see FIGS. 13). Samples being analyzed are compared to a reference sample, or samples. This comparison, or expression profile, is used, e.g., to determine if biomolecules, e.g., proteins, are present in abnormally high or low amounts compared to the reference. The determination of a difference in expression of a species in a sample relative to a reference sample is used, e.g., to diagnose disease in a patient, to determine natural variance in a population, or to determine the genotype of an individual. A comparison of protein abundances between normal and tumor cells for an individual, or across a population of patients, would be exemplary applications.

Drug Targets

Once a protein is identified in a public or private database, the gene encoding the protein is cloned and introduced into bacterial, yeast, or mammalian host cells. Where such a gene is not identified in a database, the gene encoding the protein is cloned, using a degenerate set of probes that encode an amino acid sequence of the protein as determined by the methods discussed above. Where a database contains one or more partial nucleotide sequences that encode an experimentally determined amino acid sequence of the protein, such partial nucleotide sequences (or their complement) serve as probes for cloning the gene, obviating the need to use degenerate sets.

Cells genetically engineered to express such a recombinant protein can be used in a screening program to identify other proteins or drugs that specifically interact with the recombinant protein, or to produce large quantities of the recombinant protein, e.g. for therapeutic administration.

In addition, a protein identified according to the present invention can be used to generate antibodies, for example, by administering the protein to an animal, such as a mouse, rat, or rabbit, for production of polyclonal or monoclonal antibodies using standard methods known in the art. Such antibodies are useful in diagnostic and prognostic tests and for purification of large quantities of the protein, for example, by antibody affinity chromatography. Antibodies may also be used for immunotherapy, such as might be used in the treatment of cancer.

Other Embodiments

All patents, patent applications, and publications referenced herein are hereby incorporated by reference.

What is claimed is:

1. A method of producing an Isotope Map for data from a mass spectrometric injection of a biological sample comprising:
a) performing Noise reduction and Centroiding with a Noise Reduction Module on said data from a mass spectrometric injection of a biological sample, and
b) generating an Isotope Map from the noise reduced and centroided data, thereby producing an Isotope Map.

2. The method of claim 1, wherein said biological sample is comprised of unlabeled biomolecules.

3. The method of claim 1, wherein said biological sample is comprised of underivatized biomolecules.

4. The method of claim 1, wherein said biological sample is comprised of biomolecules that are both unlabeled and underivatized.

5. The method of claim 1, wherein said biological sample is comprised of cleaved biomolecules.

6. The method of claim 5, wherein said biomolecules are cleaved with an enzyme.

7. The method of claim 6, wherein said enzyme is trypsin.

8. A method for producing a Peptide Map from an Isotope Map for data from a mass spectrometric injection of a biological sample comprising:
a) performing Peptide Detection on an Isotope Map with a Peptide Detection Module, and
b) generating a Peptide Map from the results of the peptide detection, thereby producing a Peptide Map.

9. A method for determining the functions to align Peptide Maps from a pair of Peptide Maps, each representing a mass spectrometric injection of a biological sample, comprising:
a) performing Peptide Map Alignment on a pair of Peptide Maps with a Peptide Map Alignment Module, and
b) determining a column offset between the pair of injections, and
c) determining a retention time transformation function between the pair of injections.

10. A method for Alignment of Peptide Maps from a pair of Peptide Maps, each representing a mass spectrometric injection of a biological sample, comprising performing Peptide Map Alignment on a pair of Peptide Maps with a Peptide Map Alignment Module, and thereby producing Aligned Peptide Maps.

11. A method of determining differentially and uniquely expressed biomolecules from a pair of mass spectrometric injections of biological samples, comprising:
a) producing Isotope Maps for the samples;
b) producing Peptide Maps from the Isotope Maps;
c) aligning the Peptide Maps, and
d) determining the differences between the aligned maps.

12. A method of matching biomolecules from an LC-MS injection to LC-MS-MS fragmentation spectra from comprising:
a) producing Isotope Maps for the injections;
b) producing Peptide Maps from the Isotope Maps;
c) aligning the Peptide Maps, and
d) determining the matching biomolecules.

13. A computer implemented method of producing an Isotope Map for data from a mass spectrometric injection of a biological sample comprising:
a) inputting data from a mass spectrometric injection of a biological sample, and performing Noise reduction and Centroiding with a Noise Reduction Module on said, and
b) generating an Isotope Map from the noise reduced and centroided data, thereby producing an Isotope Map.

14. The computer implemented method of claim 13, wherein said biological sample is comprised of unlabeled biomolecules.

15. The computer implemented method of claim 13, wherein said biological sample is comprised of underivatized biomolecules.

16. The computer implemented method of claim 13, wherein said biological sample is comprised of biomolecules that are both unlabeled and underivatized.

17. The computer implemented method of claim 13, wherein said biological sample is comprised of cleaved biomolecules.

18. The computer implemented method of claim 17, wherein said biomolecules are cleaved with an enzyme.

19. The computer implemented method of claim 18, wherein said enzyme is trypsin.

20. A computer implemented method for producing a Peptide Map from an Isotope Map for data from a mass spectrometric injection of a biological sample comprising:
a) inputting an Isotope Map;
b) performing Peptide Detection on said Isotope Map with a Peptide Detection Module, and
c) generating a Peptide Map from the results of the peptide detection, thereby producing a Peptide Map.

21. A computer implemented method for determining the functions to align Peptide Maps from a pair of Peptide Maps, each representing a mass spectrometric injection of a biological sample, comprising:
a) inputting a pair of Peptide Maps;
b) performing Peptide Map Alignment on a pair of Peptide Maps with a Peptide Map Alignment Module;
c) determining a column offset between the pair of injections, and
d) determining a retention time transformation function between the pair of injections.

22. A computer implemented method for Alignment of Peptide Maps from a pair of Peptide Maps, each representing a mass spectrometric injection of a biological sample, comprising inputting a pair of Peptide Maps, performing Peptide Map Alignment on said pair of Peptide Maps with a Peptide Map Alignment Module, and thereby producing Aligned Peptide Maps.

23. A computer implemented method of determining differentially and uniquely expressed biomolecules from a pair of mass spectrometric injections of biological samples, comprising:
a) inputting data from said injections;
b) producing Isotope Maps for the samples;
c) producing Peptide Maps from the Isotope Maps;
d) aligning the Peptide Maps, and
e) determining the differences between the aligned maps.

24. A computer implemented method of matching biomolecules from an LC-MS injection to LC-MS-MS fragmentation spectra from comprising:
a) inputting data from said injections;
b) producing Isotope Maps for the injections;
c) producing Peptide Maps from the Isotope Maps;
d) aligning the Peptide Maps, and
e) determining the matching biomolecules.

25. A computer-readable memory having stored thereon a program for producing an Isotope Map for data from a mass spectrometric injection of a biological sample comprising:
a) computer code that receives as input data from a mass spectrometric injection of a biological sample, and performing Noise reduction and Centroiding with a Noise Reduction Module on said, and
b) and computer code that generates an Isotope Map from the noise reduced and centroided data, thereby producing an Isotope Map.

26. The computer-readable memory of claim 25, wherein said biological sample is comprised of unlabeled biomolecules.

27. The computer-readable memory of claim 25, wherein said biological sample is comprised of underivatized biomolecules.

28. The computer-readable memory of claim 25, wherein said biological sample is comprised of biomolecules that are both unlabeled and underivatized.

29. The computer-readable memory of claim 25, wherein said biological sample is comprised of cleaved biomolecules.

30. The computer-readable memory of claim 29, wherein said biomolecules are cleaved with an enzyme.

31. The computer-readable memory of claim 30, wherein said enzyme is trypsin.

32. A computer-readable memory having stored thereon a program for producing a Peptide Map from an Isotope Map for data from a mass spectrometric injection of a biological sample comprising:
a) computer code that receives as input an Isotope Map;
b) computer code that performs Peptide Detection on said Isotope Map with a Peptide Detection Module, and
c) computer code that generates a Peptide Map from the results of the peptide detection, thereby producing a Peptide Map.

33. A computer-readable memory having stored thereon a program for determining the functions to align Peptide Maps from a pair of Peptide Maps, each representing a mass spectrometric injection of a biological sample, comprising:
a) computer code that receives as input a pair of Peptide Maps;
b) computer code that performs Peptide Map Alignment on a pair of Peptide Maps with a Peptide Map Alignment Module;
c) computer code that determines a column offset between the pair of injections, and
d) computer code that determines a retention time transformation function between the pair of injections.

34. A computer-readable memory having stored thereon a program for Alignment of Peptide Maps from a pair of Peptide Maps, each representing a mass spectrometric injection of a biological sample, comprising computer code that receives as input a pair of Peptide Maps, and computer code that performs Peptide Map Alignment on said pair of Peptide Maps with a Peptide Map Alignment Module, and thereby produces Aligned Peptide Maps.

35. A computer-readable memory having stored thereon a program for determining differentially and uniquely expressed biomolecules from a pair of mass spectrometric injections of biological samples, comprising:
a) computer code that receives as input data from said injections;
b) computer code that produces Isotope Maps for the samples;
c) computer code that produces Peptide Maps from the Isotope Maps;
d) computer code that aligns the Peptide Maps, and
e) computer code that determines the differences between the aligned maps.

36. A computer-readable memory having stored thereon a program for matching biomolecules from an LC-MS injection to LC-MS-MS fragmentation spectra from comprising:
a) computer code that receives as input data from said injections;
b) computer code that produces Isotope Maps for the injections;
c) computer code that produces Peptide Maps from the Isotope Maps;
d) computer code that aligns the Peptide Maps, and
e) computer code that determines the matching biomolecules.

37. A computer system for producing an Isotope Map for data from a mass spectrometric injection of a biological sample comprising a processor and a memory coupled to said processor, said memory encoding one or more programs, said one or more programs causing said processor to perform a method comprising:
a) computer code that receives as input data from a mass spectrometric injection of a biological sample, and performing Noise reduction and Centroiding with a Noise Reduction Module on said, and
b) computer code that generates an Isotope Map from the noise reduced and centroided data, thereby producing an Isotope Map.

38. The computer system of claim 37, wherein said biological sample is comprised of unlabeled biomolecules.

39. The computer system of claim 37, wherein said biological sample is comprised of underivatized biomolecules.

40. The computer system of claim 37, wherein said biological sample is comprised of biomolecules that are both unlabeled and underivatized.

41. The computer system of claim 37, wherein said biological sample is comprised of cleaved biomolecules.

42. The computer system of claim 41, wherein said biomolecules are cleaved with an enzyme.

43. The computer system of claim 42, wherein said enzyme is trypsin.

44. A computer system for producing a Peptide Map from an Isotope Map for data from a mass spectrometric injection of a biological sample comprising a processor and a memory coupled to said processor, said memory encoding one or more programs, said one or more programs causing said processor to perform a method comprising:
a) computer code that receives as input an Isotope Map;
b) computer code that performs Peptide Detection on said Isotope Map with a Peptide Detection Module, and
c) computer code that generates a Peptide Map from the results of the peptide detection, thereby producing a Peptide Map.

45. A computer system for determining the functions to align Peptide Maps from a pair of Peptide Maps, each representing a mass spectrometric injection of a biological sample, comprising a processor and a memory coupled to said processor, said memory encoding one or more programs, said one or more programs causing said processor to perform a method comprising:
a) computer code that receives as input a pair of Peptide Maps;
b) computer code that performs Peptide Map Alignment on a pair of Peptide Maps with a Peptide Map Alignment Module;
c) computer code that determines a column offset between the pair of injections, and
d) computer code that determines a retention time transformation function between the pair of injections.

46. A computer system for Alignment of Peptide Maps from a pair of Peptide Maps, each representing a mass spectrometric injection of a biological sample, comprising a processor and a memory coupled to said processor, said memory encoding one or more programs, said one or more programs causing said processor to perform a method comprising computer code that receives as input a pair of Peptide Maps, and computer code that performs Peptide Map Alignment on said pair of Peptide Maps with a Peptide Map Alignment Module, and thereby produces Aligned Peptide Maps.

47. A computer system for determining differentially and uniquely expressed biomolecules from a pair of mass spectrometric injections of biological samples, comprising a processor and a memory coupled to said processor, said memory encoding one or more programs, said one or more programs causing said processor to perform a method comprising:
a) computer code that receives as input data from said injections;
b) computer code that produces Isotope Maps for the samples;
c) computer code that produces Peptide Maps from the Isotope Maps;
d) computer code that aligns the Peptide Maps, and
e) computer code that determines the differences between the aligned maps.

48. A computer system for matching biomolecules from an LC-MS injection to LC-MS-MS fragmentation spectra from comprising a processor and a memory coupled to said processor, said memory encoding one or more programs, said one or more programs causing said processor to perform a method comprising:
a) computer code that receives as input data from said injections;
b) computer code that produces Isotope Maps for the injections;
c) computer code that produces Peptide Maps from the Isotope Maps;
d) computer code that aligns the Peptide Maps, and
e) computer code that determines the matching biomolecules.

49. A method for displaying information on an Isotope Map for data from a mass spectrometric injection of a biological sample comprising:
a) inputting data from a mass spectrometric injection of a biological sample;
b) performing Noise reduction and Centroiding with a Noise Reduction Module on said data;
b) generating an Isotope Map from the noise reduced and centroided data, thereby producing an Isotope Map, and
c) displaying information on said Isotope Map to a user..

50. The method of claim 49, wherein said biological sample is comprised of unlabeled biomolecules.

51. The method of claim 49, wherein said biological sample is comprised of underivatized biomolecules.

52. The method of claim 49, wherein said biological sample is comprised of biomolecules that are both unlabeled and underivatized.

53. The method of claim 49, wherein said biological sample is comprised of cleaved biomolecules.

54. The method of claim 53, wherein said biomolecules are cleaved with an enzyme.

55. The method of claim 54, wherein said enzyme is trypsin.

56. A method for displaying information on a Peptide Map produced from an Isotope Map for data from a mass spectrometric injection of a biological sample comprising:
a) inputting an Isotope Map;
b) performing Peptide Detection on said Isotope Map with a Peptide Detection Module;
c) generating a Peptide Map from the results of the peptide detection, thereby producing a Peptide Map, and
d) displaying information on said Peptide Map to a user.

57. A method for displaying information on the functions to align Peptide Maps from a pair of Peptide Maps, each representing a mass spectrometric injection of a biological sample, comprising:
a) inputting a pair of Peptide Maps;
b) performing Peptide Map Alignment on a pair of Peptide Maps with a Peptide Map Alignment Module;
c) determining a column offset between the pair of injections;
d) determining a retention time transformation function between the pair of injections, and
e) displaying information on said functions to a user.

58. A method for displaying information on Alignment of Peptide Maps from a pair of Peptide Maps, each representing a mass spectrometric injection of a biological sample, comprising inputting a pair of Peptide Maps, and performing Peptide Map Alignment on said pair of Peptide Maps with a Peptide Map Alignment Module, thereby producing Aligned Peptide Maps, displaying information on said Aligned Peptide Maps to a user.

59. A method for displaying information on differentially and uniquely expressed biomolecules from a pair of mass spectrometric injections of biological samples, comprising:
a) inputting data from said injections;
b) producing Isotope Maps for the samples;
c) producing Peptide Maps from the Isotope Maps;
d) aligning the Peptide Maps;
e) determining the differences between the aligned maps, and
f) displaying information on said differences to a user.

60. A method for displaying information on matching biomolecules from an LC-MS injection to LC-MS-MS fragmentation spectra from comprising:
a) inputting data from said injections;
b) producing Isotope Maps for the injections;
c) producing Peptide Maps from the Isotope Maps;
d) aligning the Peptide Maps, and
e) determining the matching biomolecules,
f) and displaying information on said matching biomolecules to a user.
